(19) United States
(12) Patent Application Publication
(10) Pub. No.: US 2023/0175077 A1
(43) Pub. Date: Jun. 8, 2023

(54) DIAGNOSTIC SYSTEMS AND METHODS FOR THE ENRICHMENT OF MICROBIAL NUCLEIC ACIDS AND THE IDENTIFICATION OF MICROORGANISMS AND/OR RESISTANCE GENES BY IMMOBILIZED ADSORPTION

(71) Applicant: CENTERS FOR DISEASE CONTROL, MINISTRY OF HEALTH AND WELFARE, Taipei City (TW)

(72) Inventors: Chien-Shun CHIOU, Taichung City (TW); Hui-Yung SONG, Taichung (TW); Bo-Han CHEN, Taichung (TW); Yu-Ping HONG, Taichung (TW); Min-Chi LU, Taichung (TW); Hui-Ling TANG, Taichung City (TW)

(73) Assignee: CENTERS FOR DISEASE CONTROL, MINISTRY OF HEALTH AND WELFARE, Taipei City (TW)

(21) Appl. No.: 18/062,181

(22) Filed: Dec. 6, 2022

Related U.S. Application Data
(60) Provisional application No. 63/286,548, filed on Dec. 6, 2021.

(30) Foreign Application Priority Data
Jun. 7, 2022 (TW) 111121091

(51) Int. Cl. C12Q 1/689; B01L 3/00

(52) U.S. Cl. CPC C12Q 1/689; B01L 3/502753; C12Q 1/6806

(57) ABSTRACT

Provided is a diagnostic system for identifying target microorganisms and/or resistance genes in a sample, including a cell lysis unit, a target nucleic acid enriching unit, a sequencing unit, and a sequence analyzing unit, wherein the cell lysis unit is configured to lyse non-target cells in the sample, the target nucleic acid enriching unit equipped with an immobilized adsorption device is configured to deplete nucleic acids of the non-target cells and to enrich nucleic acids of the target microorganisms, and the sequencing unit and the sequence analyzing unit are configured to produce identification results of the microbial species and/or resistance genes from the sequences of the enriched nucleic acids. Also provided is a method for enriching target nucleic acids in a sample and a method for identifying target microorganisms and/or resistance genes by sequencing the enriched nucleic acids of the target microorganisms.

### TECHNICAL FIELD

The present disclosure relates to diagnostic systems and methods for depleting non-target (e.g., human, animal, and plant) nucleic acids from a sample to enrich target (e.g., microbial) nucleic acids by immobilized adsorption, and also relates to diagnostic systems and methods for identifying target microorganisms and/or resistance genes from the sequences of the enriched target nucleic acids.

### BACKGROUND

Rapid and accurate recognition of pathogens and antimicrobial resistance is crucial for improving patient health. Currently, the “gold standard” method for clinical diagnostics is based on phenotypic analysis of microbial culture. However, this diagnostic process takes at least 24 hours to serval days to obtain a preliminary answer from bacterial growth and tests in a clinical microbial laboratory. This cannot generate timely guidance in the initial stage for a patient against infectious diseases, such as bacteremia, sepsis, and pneumonia, which may quickly become deteriorative and life-threatening. Accordingly, the patient suffering from sepsis is faced with ineffective or excessive antibiotic treatment, and that could lead to the emergence of multidrug-resistant pathogens due to inappropriate use of antibiotics.

The typically applicable technology for rapid detection of pathogens is nucleic acid amplification technology (NAAT), and it has been applied in, for example, the diagnosis of sepsis [e.g., Septifast (Roche Diagnostics, Mannheim, Germany)] and the respiratory tract infection [e.g., FilmArray Respiratory Panel (Biofire Defense, Salt Lake City, USA)]. Nevertheless, NAAT is limited by primer design, such that the detection of different target pathogens and resistance genes can only be performed in different reactions. Taking the FilmArray Blood Culture Identification (BCID) Panel for example, only 33 specific target pathogens and 10 specific resistance genes can be detected thereby. Therefore, most pathogens and resistance genes would not be applicable; particularly, rare pathogens and special resistance genes would be hardly identifiable, such that the traditional microbiological culture cannot be completely replaced with NAAT. There is thus still an urgent need for a universal diagnostic technology that can rapidly identify pathogens (such as viruses, bacteria, and fungi) and resistance genes as many as possible.

Recently, next-generation DNA sequencing (NGS), including Illumina, PacBio, and Nanopore sequencing platforms, has widely been used to obtain DNA sequences for accurate identification of pathogens and resistance genes, and for other applications, such as genotyping. However, the application of NGS in the identification of pathogens and resistance genes is faced with a big challenge as clinical specimens or blood cultures usually contain a large amount of non-target (e.g., human, animal, and plant) nucleic acids. It means that only a very small amount of the sequences generated from NGS can be used in the identification of pathogens and resistance genes, which may lead to low sensitivity in detecting pathogens due to the low abundance of target DNA sequences. Also, filtering out host sequences from a large amount of raw data is time-consuming and highly dependent on computational capability.

Nowadays, several approaches have been developed for the depletion of non-target nucleic acids in specimens. MolYsis Basic 5 Kit (Molzym, Bremen, Germany) utilizes a nuclease to digest non-target nucleic acids, while the extracted nucleic acid fragments of bacteria are relatively short, and thus it would be difficult to generate long sequence reads. NEBNext Microbiome DNA Enrichment Kit (New England Biolabs, Inc., USA) utilizes a monoclonal antibody capable of specifically binding the methylated CpG island of the human genome; however, DNA methylation is unevenly distributed across the human genome, and this kit is not cost-effective for routine examination. QIAamp BiOstic Bacteremia DNA Kit (QIAGEN, Hilden, Germany) utilizes multiple centrifugation steps to separate host cells according to the difference in cell density. However, there is still an unmet need to provide a fast and cost-effective strategy for the identification of pathogens as well as features associated with antibiotic resistance in a clinical setting and general microbiological laboratories.

### SUMMARY

In view of the foregoing, the present disclosure provides a diagnostic system and a method for depleting non-target nucleic acids from specimens by immobilized adsorption, thereby enriching target nucleic acids therein. The diagnostic system and the method provided herein have a variety of applications, including, for example, the identification of bacterial species and resistance genes through the pretreatment of a biological sample obtained from a host.

In at least one embodiment of the present disclosure, a method for enriching a target (e.g., bacterial) nucleic acid in a sample is provided. The method comprises providing a sample including a target microorganism and a non-target cell that originate from different species; adding a non-ionic surfactant to the sample to lyse the non-target cell and release a non-target nucleic acid from the non-target cell; contacting the sample with a solid phase adsorbent to bind free nucleic acids (including the non-target nucleic acid) in the sample; and removing the solid phase adsorbent and the nucleic acids thereon, thereby enriching the target nucleic acid contained in the target microorganism in the sample.

In at least one embodiment of the present disclosure, a diagnostic system for identifying a target microorganism and/or a resistance gene in a sample is provided. The diagnostic system comprises a cell lysis unit configured to lyse a non-target cell in the sample, wherein the target microorganism and the non-target cell originate from different species; a target nucleic acid enrichment unit equipped with an immobilized adsorption device and configured to deplete a nucleic acid of the lysed non-target cell, thereby enriching a nucleic acid of the target microorganism in the sample; a sequencing unit configured to sequence the enriched nucleic acid of the target microorganism; and a sequence analysis unit connected to the sequencing unit and configured to receive sequencing data generated by the sequencing unit and to compare the sequencing data with a microbial genome database and/or a resistance gene database, thereby producing an identification result of the target microorganism and/or the resistance gene carried by the target microorganism.

In at least one embodiment of the present disclosure, the immobilized adsorption device comprises a solid phase adsorbent, and the cell lysis unit comprises a non-ionic surfactant. In some embodiments, the lysis of the non-target cell is performed in an alkaline environment. In some embodiments, the solid phase adsorbent used in the present disclosure does not contain an antibody. In some embodiments, the binding or removal of non-target nucleic acids or free nucleic acids in the sample by the immobilized adsorption device is not based on the principle of antibody-antigen interaction.

In at least one embodiment of the present disclosure, the diagnostic system further comprises a target microorganism amplification unit configured to amplify an amount of the target microorganism or a nucleic acid thereof. In some embodiments, the target microorganism amplification unit comprises a blood culture device.

In at least one embodiment of the present disclosure, the sequencing unit is at least one of a next-generation sequencing platform, a high-throughput sequencing platform, an Illumina sequencing platform, a Nanopore sequencing platform, a PacBio sequencing platform, and a Sanger sequencing platform.

In at least one embodiment of the present disclosure, in order to identify microorganisms and resistance genes and/or predict antimicrobial resistance (AMR) of the microorganisms, the sequencing data to be compared are subjected to the following procedures through the microorganism comparison software and/or the resistance gene interpretation software: obtaining an index of the indicated length sequence in the sequencing data to be compared; correcting and assembling the microbial genome and bacterial plasmid sequences; reading the corresponding sequence from the reference gene sequence according to the index; and determining whether the corresponding sequence and the sequencing data to be compared are the same or not, thereby producing an identification result.

In at least one embodiment of the present disclosure, the sequence analysis unit is further configured to analyze the resistance gene carried by the target microorganism, e.g., an antimicrobial resistance gene. In some embodiments, the sequence analysis unit is further configured to calculate at least one parameter selected from the number of effective sequences for alignment, coverage, coverage depth, relative abundance, and degree of dispersion, thereby producing the identification result of the target microorganism and/or the resistance gene carried by the target microorganism.

In at least one embodiment of the present disclosure, the sequence analysis unit generates sequencing data with at least 20 times the genome size of the target microorganism. In some embodiments, the sequencing data that are generated by the sequence analysis unit within, for example, 15 min throughput or have at least one time the genome size of the target microorganism are used to calculate the distribution of the microorganism greater than 1% of the total sequence reads, as the basis for the relative abundance of the target microorganism in the sample. In some embodiments, the sequence analysis unit is further configured to detect complete resistance genes, the subtypes thereof, and resistance-relevant mutations in the target microorganism within, for example, 6 hours, thereby predicting antimicrobial resistance of the target microorganism.

In at least one embodiment of the present disclosure, a method for using the diagnostic system is also provided. The method comprises providing a sample including a target microorganism and a non-target cell that originate from different species; lysing the non-target cell by the cell lysis unit; depleting free nucleic acids, especially the non-target nucleic acid released from the non-target cell, in the sample by the target nucleic acid enrichment unit, thereby enriching a target nucleic acid of the target microorganism in the sample; sequencing the enriched nucleic acid by the sequencing unit; and producing an identification result of the target microorganism and/or the resistance gene carried by the target microorganism by the sequence analysis unit.

In at least one embodiment of the present disclosure, the lysis of the non-target cell comprises adding the non-ionic surfactant to the sample by the cell lysis unit. In some embodiments, the depletion of the free nucleic acids comprises contacting the sample with the solid phase adsorbent by the target nucleic acid enrichment unit, and removing the solid phase adsorbent and the free nucleic acids thereon, thereby enriching the target nucleic acid in the sample.

In at least one embodiment of the present disclosure, a method for enriching a target nucleic acid in a sample is also provided. The method comprises providing a sample including a target microorganism and a non-target cell that originate from different species; lysing the non-target cell by a cell lysis unit of a diagnostic system to release a non-target nucleic acid from the non-target cell, and depleting the non-target nucleic acid by a target nucleic acid enrichment unit of the diagnostic system, thereby enriching the target nucleic acid of the target microorganism in the sample. In some embodiments, the target nucleic acid enrichment unit of the diagnostic system comprises an immobilized adsorption device containing a solid phase adsorbent. In some embodiments, the depletion of the non-target nucleic acid comprises contacting the sample with the solid phase adsorbent to bind the free nucleic acids, and removing the solid phase adsorbent, thereby enriching the target nucleic acids in the sample.

In at least one embodiment of the present disclosure, the method further comprises sequencing the enriched nucleic acid by a sequencing assay to generate sequencing data, and comparing the sequencing data with a microbial genome database and/or a resistance gene database, thereby producing an identification result of the target microorganism and/or the resistance gene carried by the target microorganism.

In at least one embodiment of the present disclosure, the solid phase adsorbent is selected from the group consisting of a silica magnetic bead, a silica bead, a column extraction membrane, an alkyl-bonded silica gel, a biochar, a cellulose, an anion exchange resin, and any combination thereof. The hydrogen bonding, hydrophobic interactions, and electrostatic interactions between the cationic portion of the adsorbent and the negatively charged phosphate groups of nucleic acids may be the driving force for the binding. In some embodiments, the solid phase adsorbent may be a silica magnetic bead or based on a silica magnetic bead. In some embodiments, the solid phase adsorbent may be controlled by salts and pH value; for example, the solid phase adsorbent may bind nucleic acids in an alkaline environment. In some embodiments, the surface of the silica magnetic bead may be further modified with a silane-modified polymer, including but not limited to tetramethoxysilane (TMOS), tetraethoxysilane (TEOS), and 3-aminopropyltriethoxysilane (APTES). In some embodiments, the solid phase adsorbent used in the present disclosure does not contain an antibody. In some embodiments, the method of the present disclosure does not include binding or removing non-target nucleic acids or free nucleic acids in the sample based on the principle of antibody-antigen interaction.

In at least one embodiment of the present disclosure, the non-ionic surfactant is selected from the group consisting of saponin, Tween, Triton, polyoxyethylene (10) oleyl ether (e.g., BrijO10), polyol, a polyoxyethylene-polyoxypropylene copolymer, polyoxyethylene ether, alkyl ethanolamide, glucoside, fatty alcohol, and any combination thereof. In some embodiments, the method further comprises incubating the non-ionic surfactant and the sample under an alkaline condition to separate the non-target nucleic acid from the non-target cell.

In at least one embodiment of the present disclosure, the target nucleic acid comprises at least one of a pathogenic nucleic acid, a microbial nucleic acid, a bacterial nucleic acid, a viral nucleic acid, a fungal nucleic acid, an algae nucleic acid, a protozoan nucleic acid, and a parasitic nucleic acid. In some embodiments, the target nucleic acid may be a bacterial nucleic acid. In some embodiments, the target nucleic acid may originate from a bacterium, e.g., an antibiotic-resistant bacterium. In some embodiments, the target nucleic acid may be a bacterial plasmid or a fragment thereof, e.g., a resistance gene.

In at least one embodiment of the present disclosure, the non-target cell is a eukaryotic host, such as an animal host. In some embodiments, the non-target nucleic acid originates from an animal host. In some embodiments, the animal host is a mammalian host. In some embodiments, the sample comprises a mammalian host nucleic acid and a nucleic acid originating from a pathogen in the mammalian host. In some embodiments, the sample is obtained from a human host and comprises a human host nucleic acid and a non-human nucleic acid.

In at least one embodiment of the present disclosure, the sample may be an environmental sample obtained from dust, soil, water, air, artificial water system, food, and the like. In some embodiments, the sample may be a biological sample obtained from a host suffering or suspected of suffering from an infectious disease. In some embodiments, the infectious disease includes, but is not limited to, bacteremia, sepsis, and pneumonia.

In at least one embodiment of the present disclosure, a method for identifying a target microorganism and/or a resistance gene in a biological sample is also provided. In some embodiments, the method of the present disclosure comprises providing the biological sample from a subject infected or suspected of being infected by the pathogen, adding a non-ionic surfactant to the biological sample, contacting the biological sample with a solid phase adsorbent to bind a non-target nucleic acid originating from the subject, removing the solid phase adsorbent, thereby enriching a nucleic acid of the pathogen in the biological sample, and sequencing the enriched nucleic acid of the pathogen by a sequencing assay.

In at least one embodiment of the present disclosure, the biological sample is selected from the group consisting of blood, serum, plasma, urine, sputum, saliva, cerebrospinal fluid, interstitial fluid, mucous, sweat, stool extract, fecal matter, synovial fluid, tears, semen, peritoneal fluid, nipple aspirates, milk, vaginal fluid, and any combination thereof.

In at least one embodiment of the present disclosure, depending on the amount of target nucleic acids in the biological sample, the method provided herein may further comprise preferentially amplifying the target microorganism, the pathogen, the target nucleic acid, and/or the nucleic acid of the pathogen in the biological sample before the addition of the non-ionic surfactant. For example, the biological sample is a blood sample that is obtained from a subject suffering from sepsis and has been preferentially subjected to blood culture. In some embodiments, the sample suitable to the method of the present disclosure may be a blood culture sample identified as positive by the continuous monitoring blood culture system (such as a blood sample identified as containing microorganisms by the Gram staining process). In some embodiments, the method provided herein further comprises removing a red blood cell from the blood sample.

In at least one embodiment of the present disclosure, the sequencing assay is selected from the group consisting of a next-generation sequencing assay, a high-throughput sequencing assay, an Illumina sequencing assay, a Nanopore sequencing assay, a PacBio sequencing assay, a Sanger sequencing assay, and any combination thereof. In some embodiments, the sequencing assay may be a Nanopore sequencing assay.

In at least one embodiment of the present disclosure, the target nucleic acid or the nucleic acid of the pathogen enriched by the method provided herein has at least 2,000 nucleotides (nt) in length. For example, the enriched target nucleic acid or the enriched nucleic acid of the pathogen to be sequenced has at least 2,000 nt, at least 2,500 nt, at least 3,000 nt, at least 3,500 nt, at least 4,000 nt, at least 4,500 nt, at least 5,000 nt, at least 5,500 nt, at least 6,000 nt, at least 6,500 nt, or at least 7,000 nt in length.

In at least one embodiment of the present disclosure, the method provided herein results in at least a 10-fold enrichment of the target nucleic acid or the nucleic acid of the pathogen originally comprised within the biological sample. For example, the method results in at least a 10-fold, at least a 10²-fold, at least a 10³-fold, at least a 10⁴-fold, or at least a 10⁵-fold enrichment of the target nucleic acid or the nucleic acid of the pathogen originally comprised within the biological sample. In some embodiments, with the enrichment method provided herein, the target nucleic acid or the nucleic acid of the pathogen accounts for more than 50%, e.g., more than 55%, more than 60%, more than 65%, more than 70%, more than 75%, more than 80%, more than 85%, more than 90%, more than 95%, and more than 99%, in the biological sample, based on the total amount of nucleic acids therein.

In at least one embodiment of the present disclosure, the method provided herein further comprises extracting the enriched nucleic acid of the pathogen from the biological sample prior to the sequencing. In some embodiments, the method provided herein further comprises identifying a resistance gene carried by the pathogen based on a sequencing result. In some embodiments, identifying the resistance gene is performed at least 20 times (such as at least 25 times, at least 30 times, at least 40 times, at least 50 times, at least 60 times, and at least 70 times) the genome size of the pathogen.

In at least one embodiment, the diagnostic system and the method of the present disclosure are effective in selectively depleting a non-target nucleic acid (e.g., a host nucleic acid) and providing high-quality pathogenic DNA that may be subjected to rapid sequencing, thereby generating long sequence reads for assembling the entire genome of the pathogen. Hence, the present disclosure is useful in eliminating the interference of non-target nucleic acids as well as accelerating and improving the bioinformatics analysis to effectively identify the species of pathogens and the resistance genes thereof.

### BRIEF DESCRIPTION OF THE DRAWINGS

For a full understanding of this disclosure, reference should be made to the following detailed descriptions, taken in connection with the accompanying drawings.

FIG. 1 is a diagram showing the diagnostic system according to at least one embodiment of the present disclosure.

FIG. 2 is a diagram showing the method for enriching a microbial nucleic acid according to at least one embodiment of the present disclosure.

FIG. 3 is a flowchart showing the operation steps of the diagnostic system according to at least one embodiment of the present disclosure.

FIGS. 4A and 4B are the distribution diagram showing the proportion of the host and target bacterial nucleic acids in the blood culture sample containing Klebsiella pneumoniae (K. pneumoniae) (FIG. 4A) or Staphylococcus aureus (S. aureus) (FIG. 4B) pretreated with the method of the present disclosure or the commercially available kits. Ctrl: control group, without pretreatment; Molysis: MolYsis Basic 5 Kit; NEB: NEBNext Microbiome DNA Enrichment Kit; QiaBB: QIAamp BiOstic Bacteremia DNA Kit; TCDC: the method of the present disclosure; H. sapiens: Homo sapiens.

FIGS. 5A and 5B show the relationship between the Nanopore reading time and the number of the identified resistance genes in the blood culture sample containing Klebsiella pneumoniae (K. pneumoniae) (FIG. 5A) or Staphylococcus aureus (S. aureus) (FIG. 5B) pretreated with the method of the present disclosure or the commercially available kits. Ctrl: control group; NEB: NEBNext Microbiome DNA Enrichment Kit; QiAamp BB: QIAamp BiOstic Bacteremia DNA Kit; TCDC: the method of the present disclosure.

FIG. 6 shows the relationship between the Nanopore reading time and the number of the identified resistance genes in the clinical samples pretreated with the method of the present disclosure.

FIG. 7 shows the comparison of the turnaround time required by the conventional blood culture, FilmArray panel, and the method of the present disclosure (TCDC). ID: bacterial identification; AST: antimicrobial susceptibility testing; AMR: identification of antimicrobial resistance gene.

### DETAILED DESCRIPTION

The description discloses some embodiments in such detail that a person skilled in the art can utilize the embodiments based on the disclosure. Not all steps or features of the embodiments are discussed in detail, as many of the steps or features will be obvious to a person skilled in the art based on this disclosure.

As used in this disclosure, the singular forms “a,” “an,” and “the” include plural referents unless the content clearly dictates otherwise. As used herein, the term “and” is intended to be inclusive unless otherwise indicated. As used herein, the term “or” is generally employed in its sense including “and/or” unless the context clearly dictates otherwise.

As used herein, the term “about” refers to a degree of deviation for a property, composition, amount, value, or parameter as identified, such as deviations based on experimental errors, measurement errors, approximation errors, calculation errors, standard deviations from a mean value, routine minor adjustments, and so forth.

As used herein, the terms “comprising,” “having,” “including,” and “containing” are to be construed as open-ended terms (i.e., meaning “including, but not limited to”) unless otherwise noted.

The present disclosure is directed to a method for enriching a target nucleic acid in a sample, e.g., a biological sample obtained from a host suffering or suspected of suffering from an infectious disease. In at least one embodiment, the sample comprises a non-target nucleic acid originating from the host and a target nucleic acid originating from a non-host source. In at least one embodiment, the method increases a ratio of the target nucleic acid relative to the non-target nucleic acid in the sample by at least 10 folds.

As used herein, the terms “patient,” “host” and “subject” are used interchangeably. The term “subject” means a human or an animal. Examples of the subject include, but are not limited to, human, monkey, mice, rat, woodchuck, ferret, rabbit, hamster, cow, horse, pig, deer, dog, cat, fox, wolf, chicken, emu, ostrich, and fish. In some embodiments, the subject is a mammal, e.g., a primate such as a human.

As used herein, the term “biological sample” refers to a sample to be processed or analyzed by any of the methods described herein that can be of any type of sample obtained from a subject to be detected. The biological samples used herein include, but are not limited to: tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as those obtained by lysing cells and separating the components thereof by centrifugation or otherwise). Other examples of biological samples include, but are not limited to, body fluid samples, such as blood, serum, plasma, urine, sputum, saliva, cerebrospinal fluid, interstitial fluid, mucous, sweat, stool extract, fecal matter, synovial fluid, tears, semen, peritoneal fluid, nipple aspirates, milk, vaginal fluid, or any combination thereof. In some embodiments, a blood sample can be whole blood or a faction thereof, e.g., serum or plasma, heparinized or EDTA treated to avoid blood clotting.

The method of the present disclosure comprises adding a non-ionic surfactant, e.g., saponin, to a sample, e.g., a biological sample comprising a host nucleic acid and a non-host nucleic acid. In at least one embodiment, the host nucleic acid and the non-host nucleic acid are contained in a cell or a particle originating from the host and a non-host source, respectively. In at least one embodiment, the non-ionic surfactant selectively causes lysis of the host cell and the interior membrane thereof, releasing a host nucleic acid, such that the host nucleic acid can be partially or completely bound to a solid phase adsorbent. The nucleic acid within a non-host cell or particle (e.g., pathogen) is essentially left intact, and would not be significantly removed from the biological sample, such that such nucleic acid can be subsequently collected and analyzed by, e.g., sequencing. The non-host nucleic acid processed or analyzed by any of the methods described herein has an average length sufficiently long to be identifiable; that is, the sequence and/or biological origin thereof can thus be ascertained. In at least one embodiment, the non-host nucleic acid enriched by the methods described herein may have at least 2,000 nucleotides in length.

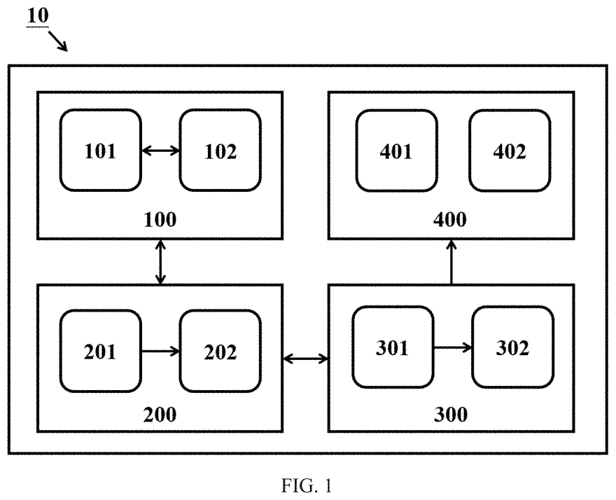

Referring to FIG. 1, this diagram illustrates the diagnostic system according to at least one embodiment of the present disclosure. The diagnostic system 10 of the present disclosure comprises a cell lysis unit 100, a target nucleic acid enrichment unit 200, a sequencing unit 300, and a sequence analysis unit 400. The cell lysis unit 100 may include a sample container 101 and a non-ionic surfactant 102 disposed toward the sample container 101, wherein the non-ionic surfactant 102 is configured to lyse a non-target cell and release a non-target nucleic acid from the lysed non-target cell. For example, after the culture, the blood sample collected from the human host may be introduced from the blood culture bottle into a centrifuge tube containing the non-ionic surfactant through a three-way sample extraction device.

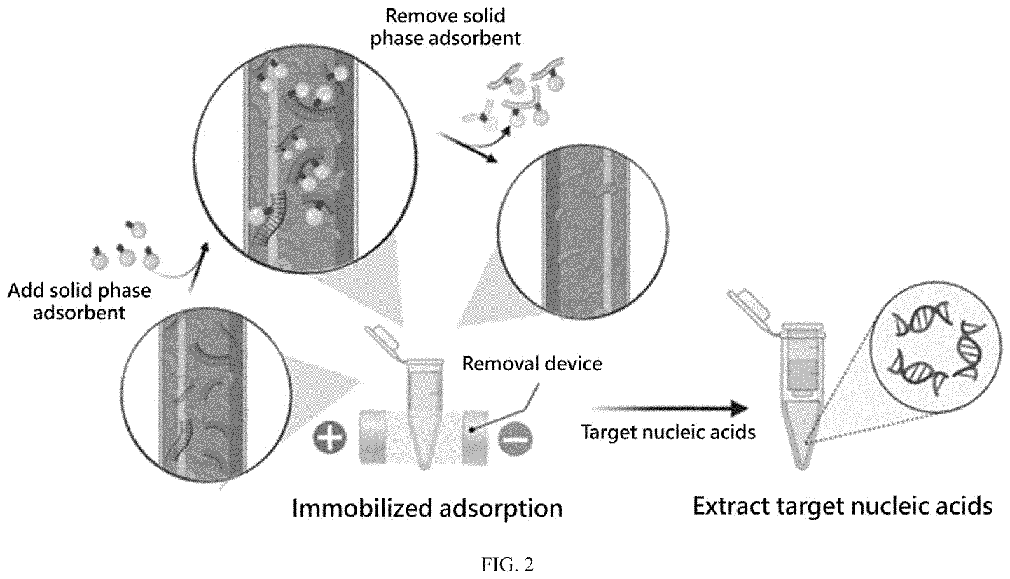

In at least one embodiment of the present disclosure, the target nucleic acid enrichment unit 200 may be connected to the cell lysis unit 100 and configured to receive the sample where the cells therein have been lysed by the cell lysis unit 100. The target nucleic acid enrichment unit 200 may include an immobilized adsorption device 201 and a nucleic acid extraction device 202, wherein the immobilized adsorption device 201 includes a solid phase adsorbent, which is configured to bind and remove the non-target nucleic acid released from the lysed cells, thereby enriching the target nucleic acid contained in the sample. The enriched target nucleic acid may be subsequently extracted by the nucleic acid extraction device 202. For example, further referring to FIG. 2, the solid phase adsorbent (such as silica magnetic beads) may be added into the sample to bind the free nucleic acids in the sample, which are then removed by a removal device (such as a magnet rack) or by using density gradient centrifugation. Therefore, the target microorganism is left in the sample, and the nucleic acid thereof can be then extracted.

Referring to FIG. 1 again, in at least one embodiment of the present disclosure, the sequencing unit 300 may be connected to the target nucleic acid enrichment unit 200 and configured to receive the nucleic acids of the target microorganism enriched by the target nucleic acid enrichment unit 200. In at least one embodiment of the present disclosure, the sequencing unit 300 may include a DNA library preparation kit 301 and a sequencer 302 for sequencing the nucleic acids of the target microorganism. In at least one embodiment, the examples of the sequencer suitable for the diagnostic system of the present disclosure include, but are not limited to, Flongle sequencer and MinION sequencer.

In at least one embodiment of the present disclosure, the sequence analysis unit 400 may be connected to the sequencing unit 300 and configured to receive the sequencing data generated by the sequencing unit 300, wherein the sequencing data include the barcode of subsequence with indicated length in the sequence to be compared (i.e., the nucleic acid sequence of the target microorganism). In at least one embodiment of the present disclosure, the sequence analysis unit 400 may include a microorganism identification module 401 and a resistance gene identification module 402. By the microorganism identification module 401, the sequencing data are compared with a microbial genome database, thereby producing the identification result of the target microorganism. Further, the resistance gene identification module 402 can be used to identify the resistance gene carried by the target microorganism.

In at least one embodiment of the present disclosure, for determining whether the sequencing data and the reference sequence of the microbial genome database are the same or not, the corresponding sequence to be compared can be read from the reference sequence according to the barcode of the sequencing data, and then the base pairs in the sequence to be compared are aligned to the reference sequence to determine whether the bases in the sequence to be compared and the reference sequence are the same or not. If the alignment result is the same, the index is used as the position information of the sequence to be compared. If the alignment result is different, it is determined that there is an inserted or deleted base pair in the sequence to be compared. In at least one embodiment, the microbial genome database suitable to the diagnostic system of the present disclosure includes, but is not limited to, Centrifuge and Karken2, which are clinical pathogen databases used to compare with bacteria, viruses, fungi, parasites, and the like.

In at least one embodiment of the present disclosure, the database for species identification includes a pathogen genome database and a pathogen literature database, whose original data sources may be a public database, such as National Center for Biotechnology Information (NCBI). At present, the microbial genome database records the reference sequences of a total of 69,836 species, including a total of 5,527 species of bacteria and archaea, 1,677 species of viruses, 5,523 species of fungi, and 865 species of parasites, as well as 62,602 species of eukaryotes. In at least one embodiment of the present disclosure, the database for resistance gene identification may be the resistance gene database Resfinder 4.0 (Center for Genomic Epidemiology, DTU, Denmark). Currently, the resistance gene database includes reference sequences with a total of 2,690 resistance genes on plasmids and 266 resistance gene mutation sites on chromosomes, and further includes 57 drugs for predicting resistance of microorganisms.

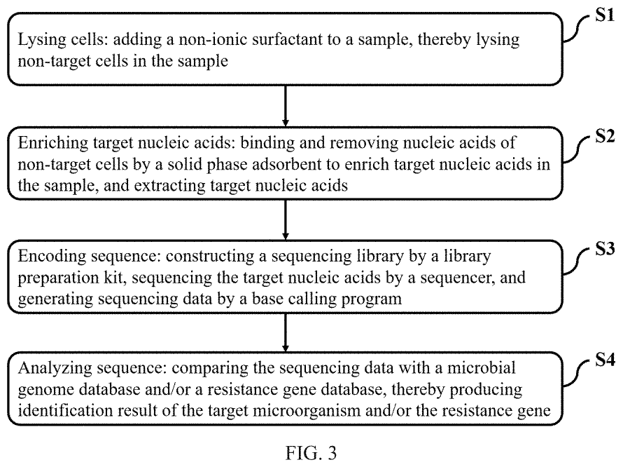

Further referring to FIG. 3, this flowchart illustrates the operation steps of the diagnostic system according to at least one embodiment of the present disclosure. The main steps S1 to S4 are lysing cells (S1), enriching target nucleic acids (S2), encoding sequence (S3), and analyzing sequence (S4). These steps are described as follows.

The step of lysing cells (S1) comprises adding a non-ionic surfactant to a sample collected from the environment or a host, thereby lysing non-target cells in the sample.

The step of enriching target nucleic acids (S2) comprises binding nucleic acids of the non-target cells by a solid phase adsorbent, and extracting target nucleic acids in the sample after removing the solid phase adsorbent.

The step of encoding sequence (S3) comprises constructing a sequencing library with a library preparation kit, sequencing the target nucleic acids by a sequencer, and generating sequencing data by a base-calling program.

The step of analyzing sequence (S4) comprises comparing the sequencing data with a microbial genome database and/or a resistance gene database, thereby producing the identification result of the target microorganism and/or the resistance gene.

The materials and processes used in the present disclosure will be provided and described in detail below.

### (1) Immobilized Adsorption of Host Nucleic Acids

When incubation of blood cultures in a system, for example, the BACTEC (BD), is flagged positive, 2 mL blood culture solution is taken and reacted with 1×red blood cell (RBC) lysis buffer at room temperature (RT) for 5 min to eliminate the RBC in the blood. Subsequently, the reacted solution is centrifuged at 3,000×g for 10 min to primarily clean the debris. The supernatant is discarded, and the pellet is resuspended with 250 μL of phosphate-buffered saline (PBS). Further, the non-ionic surfactant (e.g., saponin, Tween, Triton, polyoxyethylene (10) oleyl ether, polyols, polyoxyethylene-polyoxypropylene copolymers, polyoxyethylene ethers, alkyl ethanolamides, glucosides, and fatty alcohols) is added in the suspension. For example, 5% saponin is added to the suspension to reach the final concentration of 2.2%, and then subjected to incubation at RT for 10 min. After centrifugation at 6,000×g for 5 min, the supernatant is discarded, and the pellet is resuspended with 200 μL of PBS. To the suspension, 100 μL of solid-phase reversible immobilization (SPRI) beads are added, followed by pipetting for 5 min. Further, after standing on a magnet rack, the supernatant is collected. The supernatant is then centrifuged at 3,000×g for 3 min, and the pellet is resuspended in 200 μL of PBS.

### (2) Extraction of Bacterial DNA

To extract bacterial DNA from the pretreated pellet for Nanopore sequencing, a commercially available kit is employed generally based on protocols described in QIAamp blood and tissue genomic DNA from Qiagen manual, except that the lysozyme and lysostaphin protocol is used to reduce processing steps and turnaround time.

After DNA has been extracted, shorter DNA fragments (less than about 300 bp in length) are depleted by SPRI beads. DNA concentration is measured with a Qubit Fluorometer by using the Qubit Broad Range double-stranded DNA (dsDNA) quantification kit, which has a quantitation range of 2 ng/μL to 1,000 ng/μL. DNA purity and contamination are assessed by using a NanoDrop spectrophotometer. The suggested sample purity is A₂₆₀/A₂₃₀>2.0 and A₂₆₀/A₂₈₀>1.8.

### (3) Library Preparation for Nanopore Sequencing

The DNA concentration of the extracted sample is adjusted to 80 ng/μL, and then 5 μL of the sample (400 ng) is added with 2.5 μL of water to a final volume of 7.5 μL. The Rapid Barcoding kit (SQK-RBK004, Oxford Nanopore) is dissolved at room temperature for a subsequent experiment.

Further, 7.5 μL of the sample, 2.5 μL of each label barcode adapter 1 to 96, the sequencing adapters, and dynein are added into a 0.2 mL microcentrifuge tube. In the process of connecting the label barcode adapters, the same label barcode adapter cannot be reused within 96 consecutive samples.

The sample is placed in a PCR machine for a reaction of 30° C. for 1 min and 80° C. for 1 min, and further placed on an ice box to mix all labeled samples. Subsequently, DNA is purified by Agencourt AMPure XP magnetic beads. The magnetic beads shall be shaken well before use. Specifically, 60 μL of the magnetic beads are added to the reacted DNA solution, placed in a mixer, and inverted for 5 min. The microcentrifuge tube is stood on a magnet rack for 10 min. After the removal of the solution, the magnetic beads are washed with 70% alcohol twice. Afterward, the magnetic beads are dispersed with 25 μL of DNase-free water to dissolve DNA in water. The magnetic beads are then removed by the magnet rack to obtain a purified DNA library.

### (4) Nanopore Sequencing Data Analysis

Sequencing is performed on MinION flow cells (R9.4.1 FLO-MIN106, Oxford Nanopore). The flow cells are placed in the MinION sequencer after returning to room temperature, and the Flow Cell Priming kit is used for the sequencing. Firstly, the flush buffer (FB) and the flush tether (FLT) are returned to room temperature, and 30 μL of FLT is added to FB to form a priming mixture. Subsequently, 800 μL of the priming mixture is loaded into the flow cells via the priming port and stood for 5 min. Further, another 200 μL of the priming mixture is loaded into the priming port.

In another microcentrifuge tube, 12 μL of the prepared DNA library is added with 37.5 μL of sequencing buffer (SQB) and 25.5 μL of loading beads to form a sequencing mixture with a total volume of 75 μL. The sequencing mixture is gently pipetted to avoid the introduction of any air bubbles, and then slowly dropped into a sample port. The reagent port and the sample port were closed for performing sequencing.

The data are collected by using the MinKNOW software v4.2.4. Base calling is performed using the Guppy command line tool with barcode de-multiplexing and FASTQ file output. Adaptor sequences are trimmed from the reads using Porechop v0.2.3, which is run with barcode de-multiplexing. Only reads for which Guppy and Porechop agreed on the barcode bin are kept to reduce the risk of cross-barcode contamination. The MinKNOW platform generated sequencing data, and all sequences per file are outputted using default settings. The first output file is produced approximately 2 hours after the start of the sequencing run until 10 hours. For this work, each output file is processed separately for keeping track of the time that passes from the start of the sequencing.

### (5) Taxonomy Classification

Raw sequencing reads (≥2,000 bp) are taxonomically classified by the classification program such as Centrifuge 1.0.4 and Kraken 2 and using default settings (minimum length of partial hits min_hitlen=22; at most k=5 distinct assignments for each read; no preferred/excluded taxa) and the reference gene sequences of bacteria, archaea, virus, and human.

Specifically, based on the barcode of subsequence with indicated length in the sequence to be compared, the corresponding sequence is readout from the reference gene sequences. The generated sequencing data are classified by the clinical pathogen database of Centrifuge 1.0.4 or Kraken 2, and the sequence whose alignment length is greater than 80% of the full length of the reference sequence and the mismatched bases in the alignment region is less than or equal to 10% is kept, so as to calculate the proportion of pathogen classification. The sample is identified as containing a pathogen if the proportion of pathogen classification is greater than 1% of the total sequence reads.

### (6) Metagenomic Assembly and Antimicrobial Resistance (AMR) Genes Search

Once sequencing data have been collected, the next step is pre-processing and base calling, followed by metagenomic assembly. Various assemblers are appropriate for the assembly of long-read metagenomic data. These include long-read assemblers, such as Canu and Flye. In addition, long reads alone can be used for error correction by using Racon and Medaka, which uses neural networks to recognize and correct Nanopore homopolymer errors and generate consensus sequence, and the Homopolish, which is a method for the removal of systematic errors in Nanopore sequencing by homologous polishing software. Raw sequencing reads (≥300 bp) and assembled contigs tagged as plasmids are searched with ResFinder 4.0 databases using BLAST. Only hits with ≥90% similarity, E-value ≤10⁻⁶, and ≥60% coverage of the database entry are kept.

The assembled sequences are compared with the resistance gene database. Based on the alignment to microbial genome and resistance genes, at least one parameter selected from the number of effective sequences for alignment (i.e., the number of sequences of the species and genes for alignment between genus/species and resistance genes), coverage (i.e., the percentage of the length of the detected microbial nucleic acid sequence to the length of the genome sequence of microorganisms and resistance genes), coverage depth (i.e., the average depth of each base that is measured in the genome), relative abundance (i.e., the proportion of the detected microorganisms to the same genus/species of microorganisms in the sample), and degree of dispersion can be calculated, thereby producing the identification result.

The following examples provide various non-limiting embodiments and properties of the present disclosure.

### Example 1: Assessments of the Method of the Present Disclosure on Depletion of Non-Target Nucleic Acids

In this example, a human blood sample containing Klebsiella pneumoniae (K. pneumoniae) strain KPC160111 or Staphylococcus aureus (S. aureus) strain TUH25713455 was pretreated with the immobilized adsorption of human nucleic acids, and then subjected to quantitative polymerase chain reaction (qPCR) and Nanopore sequencing.

The results indicated that the bacterial nucleic acids were enriched in the sample with the pretreatment of immobilized adsorption. As shown in Table 1 below, in the pretreated sample, the human nucleic acids were depleted to 0.005 to 0.016 times of the control sample, while the bacterial nucleic acids were increased to 2.34 to 5.78 times of the control sample.

TABLE 1
The amounts of host and bacterial nucleic acids measured by qPCR

| | | | Duplicated | Duplicated | | | Fold |
|---|---|---|---|---|---|---|---|
| Spiked in blood | qPCR assay | Sample | 1 | 2 | Average | ΔCq | difference |
| K. pneumoniae | K. pneumoniae | Undepleted | 15.02 | 15.04 | 15.03 | 2.53 | 5.78 |
| | | Depleted | 11.21 | 13.79 | 12.50 | | |
| | Human | Undepleted | 22.81 | 22.87 | 22.84 | −7.69 | 0.005 |
| | | Depleted | 30.76 | 30.29 | 30.53 | | |
| S. aureus | S. aureus | Undepleted | 10.82 | 13.85 | 12.34 | 1.23 | 2.34 |
| | | Depleted | 9.05 | 13.17 | 11.11 | | |
| | Human | Undepleted | 19.21 | 20.13 | 19.67 | −5.97 | 0.016 |
| | | Depleted | 25.56 | 25.71 | 25.64 | | |

Cq: quantification cycle

Further, the results of Nanopore sequencing indicated that the number of reads (i.e., No. of reads), the read length (including average read length, median read length, and N50), and the total base obtained from the pretreated sample were all significantly higher than that from the control sample (Table 2).

TABLE 2
The quality of bacterial nucleic acids prepared by the method of the present disclosure
for Nanopore sequencing

| | | Average | Mean | Median | | | |
|---|---|---|---|---|---|---|---|
| | | read | read | read | | | |
| | | length | quality | length | No. of | | |
| Spiked in blood | Sample | (bp) | (Q) | (bp) | reads | N50 | Total base |
| K. pneumoniae | Undepleted | 5,613 | 13.6 | 3,038 | 84,376 | 11,470 | 473,680,706 |
| | Depleted | 9,833 | 12.8 | 6,467 | 168,409 | 17,242 | 1,656,087,432 |
| S. aureus | Undepleted | 4,774 | 13.7 | 2,570 | 13,959 | 9,765 | 66,640,453 |
| | Depleted | 8,713 | 13 | 5,536 | 111,447 | 15,610 | 971,092,018 |

N50: the sequence length of the shortest contig at 50% of the total genome length.

In terms of the proportion of bacterial nucleic acids after the Nanopore sequencing, Table 3 below shows that the proportion of non-target nucleic acids (i.e., human nucleic acids) was significantly decreased from 63.09% to 0.13% in the pretreated sample containing K. pneumoniae, and from 75.35% to 0.11% in the pretreated sample containing S. aureus; on the other hand, the proportion of bacterial nucleic acids was increased from 28.34% to 82.01% (K. pneumoniae) and from 20.72% to 81.14% (S. aureus).

TABLE 3
The proportion of bacterial nucleic acids after the Nanopore sequencing

| | | | | Human | Target | |
|---|---|---|---|---|---|---|
| | | Total | Classified | DNA | DNA | Unclassified |
| Spiked in blood | Sample | reads | reads | reads | reads | reads |
| K. pneumoniae | Undepleted | 84,376 | 81,865 | 53,234 | 23,910 | 2,511 |
| | | | | (63.09%) | (28.34%) | |
| | Depleted | 168,409 | 161,348 | 221 | 138,110 | 7,061 |
| | | | | (0.13%) | (82.01%) | |
| S. aureus | Undepleted | 13,959 | 13,573 | 10,518 | 2,904 | 386 |
| | | | | (75.35%) | (20.72%) | |
| | Depleted | 111,447 | 106,364 | 118 | 90,424 | 5,083 |
| | | | | (0.11%) | (81.14%) | |

### Example 2: Identification of Bacterial Species and Resistance Genes

In this example, a human blood sample containing K. pneumoniae or S. aureus was pretreated with the immobilized adsorption of human nucleic acids or the commercially available kits (i.e., MolYsis Basic 5 Kit, NEBNext Microbiome DNA Enrichment Kit, and QIAamp BiOstic Bacteremia DNA Kit), and then subjected to qPCR, Nanopore sequencing, and identification of the bacterial species and resistance genes based on the sequencing data generated from the Nanopore sequencing.

In comparison with the commercially available kits, the sample pretreated with the immobilized adsorption provided herein had the longest read length (including average read length and mean read length) (Table 4 and Table 5 below).

TABLE 4
Blood culture samples spiked with K. pneumoniae strain KPC160111
(having 29 resistance genes) pretreated with different methods

| | DNA | Average read | Median read | No. of | |
|---|---|---|---|---|---|
| Method | (ng/μL) | length (bp) | length (bp) | reads | Total base |
| Ctrl | 30.7 | 5,074 | 2,413 | 37,619 | 190,883,881 |
| Molysis | 5.9 | 1,438 | 187 | 2,376 | 3,416,680 |
| NEB | 14.7 | 4,223 | 2,300 | 3,765 | 1,222,820,069 |
| QiAamp BB | 28 | 2,383 | 1,618 | 342,288 | 815,884,192 |
| TCDC | 32.8 | 9,921 | 6,788 | 498,615 | 4,946,906,836 |

Ctrl: control group, in which the blood sample was not pretreated to deplete non-target nucleic acids
Molysis: MolYsis Basic 5 Kit
NEB: NEBNext Microbiome DNA Enrichment Kit
QiAamp BB: QIAamp BiOstic Bacteremia DNA Kit
TCDC: the method provided herein

TABLE 5
Blood culture samples spiked with S. aureus strain TUH25713455
(having 2 resistance genes) pretreated with different methods

| | DNA | Average read | Median read | No. of | |
|---|---|---|---|---|---|
| Method | (ng/μL) | length (bp) | length (bp) | reads | Total base |
| Ctrl | 30.6 | 2,293 | 921 | 10,549 | 24,197,739 |
| Molysis | 93.8 | 821 | 185 | 7,475 | 6,143,528 |
| NEB | 10.2 | 541 | 221 | 11,929 | 6,459,450 |
| QiAamp BB | 94.4 | 1,603 | 888 | 585,887 | 935,519,573 |
| TCDC | 11.8 | 2,618 | 1,299 | 298,892 | 782,622,266 |

Ctrl: control group, in which the blood sample was not pretreated to deplete non-target nucleic acids
Molysis: MolYsis Basic 5 Kit
NEB: NEBNext Microbiome DNA Enrichment Kit
QiAamp BB: QiAamp BiOstic Bacteremia DNA Kit
TCDC: the method provided herein

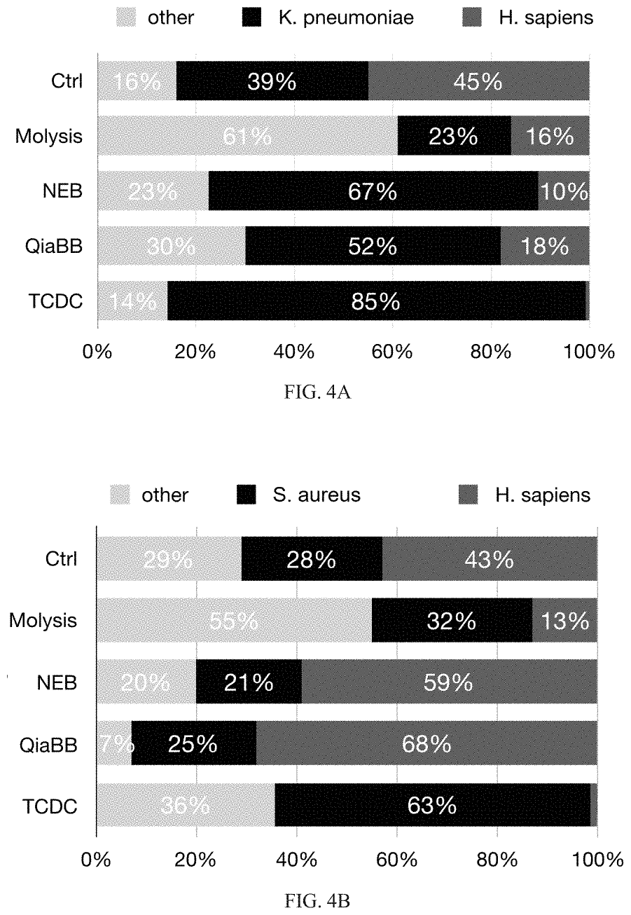

Further, as shown in FIGS. 4A and 4B, the proportion of bacterial nucleic acids in the sample pretreated with the method of the present disclosure was much higher than that pretreated with other commercially available kits. For example, the sequencing data obtained by Nanopore sequencing were used to identify the species distribution by the Centrifuge database, and the results indicated that the proportion of human nucleic acids in the sample pretreated with the method of the present disclosure was only about 1%, while the proportion of bacterial nucleic acids could account for 85% (K. pneumoniae) or 63% (S. aureus). It thus can be seen that the method of the present disclosure significantly increased the proportion of bacterial nucleic acids in the pretreated sample in comparison with the commercially available kits.

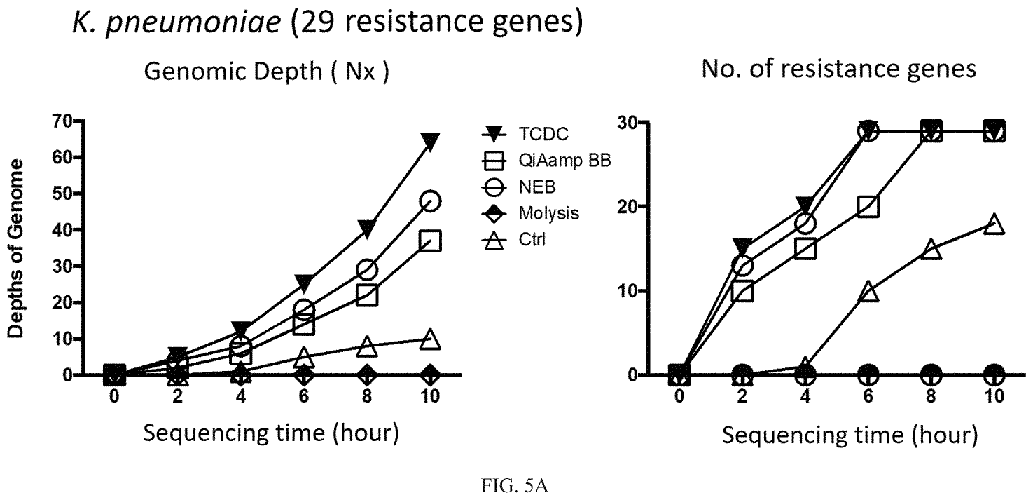

In addition, as shown in FIG. 5A, all 29 resistance genes carried by K. pneumoniae strain KPC160111 could be identified within 6-hour sequencing, indicating that with the pretreatment method provided herein, the sequence reads that reached 20× coverage depths of genome size in K. pneumoniae become enough within 6-hour sequencing to detect complete resistance genes. Similarly, FIG. 5B shows that 2 resistance genes carried by S. aureus could be identified within 2-hour sequencing which reached 20× coverage depths of genome size in S. aureus. In comparison to the QiAamp BB kit, it required 6-hour sequencing to obtain enough amount of sequence for detection, while the sequence reads obtained from the sample pretreated with NEB kit in 10 hours were still not enough to identify 2 resistance genes.

### Example 3: Assessments of Clinical Specimens on the Identification of Microbial Species and Resistance Genes

In this example, 36 human blood culture specimens provided by a hospital in Taiwan were pretreated with the immobilized adsorption of non-target nucleic acids, and then subjected to the identification of pathogens and the detection of resistance genes.

The results were shown in Table 6 below, in which the percentage represents a proportion of sequence reads. It can be found that among the 36 blood specimens, 33 cases indicated that the pathogens identified by the method of the present disclosure were consistent with those identified by the conventional microbial culture; moreover, in the case that the sample contained more than one pathogen or the pathogens therein were different species of the same genus, the minor pathogens or species in the sample could also be identified by the method of the present disclosure. Three cases, Nos. 7, 14, and 24, which showed inconsistent identification results with those obtained from the microbial culture, might be more likely to be close to the real result of infection.

TABLE 6
Comparison of conventional microbial culture and the method
of the present disclosure in terms of pathogen identification

| Sample | | Conventional | Identification using the TCDC protocol | |
|---|---|---|---|---|
| No. | G | culture method | (>1% of classified reads) | Note |
| 1 | − | Klebsiella pneumoniae | Klebsiella pneumoniae (77.7%) | |
| 2 | − | Escherichia coli | Escherichia coli (75.6%) | |
| 3 | − | Acinetobacter baumannii | Acinetobacter baumannii (62.9%) | |
| 4 | + | Staphylococcus aureus | Staphylococcus aureus (53%)/Escherichia coli (25%) | MRSA |
| 5 | − | Escherichia coli | Escherichia coli (65.6%) | |
| 6 | + | Staphylococcus aureus | Staphylococcus aureus (56%) | MRSA |
| 7 | + | Staphylococcus aureus | S. epidermidis (47%)/aureus (17%)/simulans | MRSA |
| | | | (1.1%)/L. johnsonii (3.2%)/A. urinaeequi | |
| | | | (1.8%)/K. pneumoniae (1.1%) | |
| 8 | − | Proteus mirabilis | Proteus mirabilis (58%) | |
| 9 | − | Escherichia coli | Escherichia coli (58%) | |
| 10 | − | Escherichia coli | Escherichia coli (68%) | |
| 11 | − | Escherichia coli | Escherichia coli (92.8%) | |
| 12 | − | Escherichia coli | Escherichia coli (86%)/Enterococcus faecium (1.9%) | |
| 13 | − | Pseudomonas | Pseudomonas BJP69 (55%)/putida (18.5%)/monteilii | |
| | | | (1.4%)/aeruginosa (1.1%)/E. hormaechi (1.1%) | |
| 14 | + | Staphylococcus | Staphylococcus capitis (47.8%)/hominis (25.5%)/aureus (1.41%) | |
| | | epidermidis | | |
| 15 | + | Enterococcus faecium | Enterococcus faecium (97%) | |
| 16 | − | Acinetobacter baumannii | Acinetobacter baumannii (58.0%) | CR |
| 17 | − | Acinetobacter baumannii | Acinetobacter baumannii (68.8%) | CR |
| 18 | − | Klebsiella pneumoniae | Klebsiella pneumoniae (76.7%)/variicola | CR |
| | | | (1.7%)/quasipneumoniae (1.2%)/Escherichia coli (2.6%) | |
| 19 | + | Staphylococcus aureus | Staphylococcus aureus (94%) | MRSA |
| 20 | − | Acinetobacter baumannii | Acinetobacter baumannii (61.3%)/Enterococcus | CR |
| | | | faecium (5.0%) | |
| 21 | − | Escherichia coli | Escherichia coli (90.4%) | CR |
| 22 | + | Enterococcus faecium | Enterococcus faecium (96.5%) | VRE |
| 23 | − | Klebsiella aerogenes | Klebsiella aerogenes (93%) | CR |
| 24 | − | Klebsiella pneumoniae | Klebsiella quasipneumoniae (60.9%)/pneumoniae (7.1%) | CR |
| 25 | − | Klebsiella variicola | Klebsiella variicola (86.9%) | CR |
| 26 | − | Klebsiella pneumoniae | Klebsiella pneumoniae (67.5%)/variicola (1.4%) | CR |
| 27 | − | Klebsiella pneumoniae | Klebsiella pneumoniae (75.1%)/Escherichia coli (2.9%) | CR |
| 28 | − | Klebsiella pneumoniae | Klebsiella pneumoniae (80.6%) | CR |
| 29 | − | Klebsiella pneumoniae | Klebsiella pneumoniae (67.6%)/Escherichia coli (2.0%) | CR |
| 30 | − | Klebsiella pneumoniae | Klebsiella pneumoniae (81.8%) | CR |
| 31 | − | Klebsiella pneumoniae | Klebsiella pneumoniae (74.2%)/Klebsiella variicola | CR |
| | | | (1.1%) | |
| 32 | − | Klebsiella pneumoniae | Klebsiella pneumoniae (62.4%)/Escherichia coli (1.2%) | CR |
| 33 | − | Klebsiella pneumoniae | Klebsiella pneumoniae (65.3%)/Escherichia coli (3.2%) | CR |
| 34 | − | Klebsiella pneumoniae | Klebsiella pneumoniae (81.5%)/Escherichia coli (3.2%) | CR |
| 35 | Y | Candida glabrata | Candida glabrata (55.4%)/Escherichia coli (2.2%) | |
| 36 | Y | Candida albicans | Candida albicans (51.3%)/Escherichia coli (1.2%) | |

G: Gram-positive (+); Gram-negative (−)
Y: Yeast
MRSA: methicillin-resistant Staphylococcus aureus
CR: carbapenem-resistant
VRE: vancomycin-resistant Enterococcus

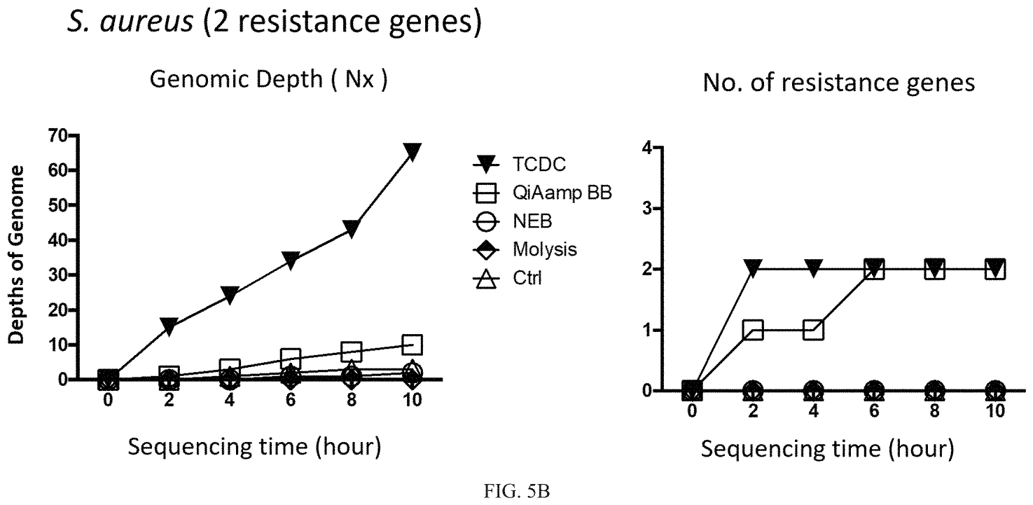

The resistance genes detected by the method of the present disclosure could be attributed to the phenotypic resistance in the sample determined by conventional antimicrobial susceptibility testing (AST). The resistance genes in samples Nos. 4, 17, 19, 21, 22, and 29 identified by the method of the present disclosure were shown in FIG. 6. It was indicated that all resistance genes carried by each sample could be identified within 2 to 6 hours of sequencing time to obtain 20× coverage depths of genome size in each pathogen.

The performance of the present disclosure (TCDC protocol) in the identification of bacterial species in 44 clinical blood specimens was compared with conventional culture, matrix-assisted laser desorption/ionization time-of-flight (MALDI-TOF) mass spectrometry, and the BIOFIRE Blood Culture Identification (BCID2, FilmArray). As shown in Table 7 below, the method of the present disclosure performed well in the identification of bacterial species in the sample containing gram-positive, gram-negative, or multiple bacteria, and had 100% consistency with the results of conventional culture MALDI-TOF. This evaluation also indicated that the method of the present disclosure was superior to FilmArray BCID2 in the identification of bacterial species in the 44 clinical specimens.

TABLE 7
Identification of bacterial species in 44 blood specimens
using the method of the present disclosure (i.e., TCDC
protocol), blood culture MALDI-TOF, and FilmArray BCID2

| | Blood Culture MALDI-TOF | | | |
|---|---|---|---|---|
| | | Number | | |
| | | of | | TCDC |
| | Bacterial species | samples | FilmArray BCID2 | Protocol |
| Gram- | Klebsiella pneumoniae | 7 | 7 | 7 |
| negative | Klebsiella variicola | 1 | Klebsiella pneumoniae* | 1 |
| | Citrobacter freundii | 2 | Enterobacteriaceae | 2 |
| | Serratia marcescens | 3 | 3 | 3 |
| | Serratia rubidaea | 1 | Enterobacteriaceae | 1 |
| | Enterobacter cloacae | 1 | 1 | 1 |
| | Moraxella osloensis | 1 | 0 | 1 |
| | Escherichia coli | 11 | 11 | 11 |
| | Pseudomonas aeruginosa | 3 | 3 | 3 |
| | Acinetobacter baumannii | 2 | 2 | 2 |
| | Acinetobacter guillouiae | 1 | 0 | 1 |
| | Stenotrophomonas maltophilia | 1 | 1 | 1 |
| | Total | 34 | 28 | 34 |
| Gram- | Staphylococcus aureus | 1 | 1 | 1 |
| positive | Group B Streptococcus | 1 | 1 | 1 |
| | Enterococcus faecium | 3 | 3 | 3 |
| | Total | 5 | 5 | 5 |
| Multiple | Escherichia coli | 1 | 1 | 1 |
| bacteria | Klebsiella pneumoniae | | | |
| | Enterococcus gallinarum | | | |
| | Klebsiella pneumoniae | 1 | 1 | 1 |
| | Enterobacter cloacae | | | |
| | Enterococcus gallinarum | 1 | 1 | 1 |
| | Candida albicans | | | |
| | Proteus mirabilis | 1 | 1 | 1 |
| | Klebsiella pneumoniae | | | |
| | Staphylococcus epidermidis | | | |
| | Klebsiella aerogenes | 1 | Klebsiella aerogenes | 1 |
| | Citrobacter cronae | | Escherichia coli | |
| | Total | 5 | 4 | 5 |

*Inconsistent result is given by the species name.

As to the clinical specimens from intensive care units, the performance of the method of the present disclosure was also compared with conventional culture MALDI-TOF, FilmArray BCID2, and Nanopore sequencing of 16S rRNA gene, and the results of pathogens identification were shown in Table 8 below. The method of the present disclosure had concordant results with the culture method in the specimens identified with one pathogen, expect that in specimen ICU2-1, the method of the present disclosure further identified additional bacterial species. The FilmArray BCID2 panel failed to identify Moraxella osloensis in specimen ICU04 and Acinetobacter guillouiae in specimen ICU13. In specimens ICU2-1 and ICU38, the FilmArray BCID2 panel could not specifically identify the species of bacteria (e.g., Citrobacter freundii).

TABLE 8
Comparison of pathogen identification between the method of the present disclosure (i.e., TCDC protocol),
blood culture MALDI-TOF, FilmArray DCID2, and Nanopore sequencing of 16S rRNA

| Sample | TCDC Protocol | | Blood Culture | | | |
|---|---|---|---|---|---|---|
| No. | (>1% of classified reads) | | MALDI-TOF | FimArray BCID2 | Nanopore-seq 16S rRNA | |
| ICU2-1 | Citrobacter freundii | 90% | Citrobacter freundii | Enterobacterales | Citrobacter | 40% |
| | complex | | | | murliniae | |
| | Citrobacter freundii | 53% | | | Citrobacter gillenii | 23% |
| | Citrobacter | 24% | | | Citrobacter freundii | 18% |
| | portucalensis | | | | | |
| | Citrobacter youngae | 3% | | | Citrobacter braakii | 16% |
| | Escherichia coli | 2% | | | | |
| ICU4 | Moraxella osloensis | 53% | Moraxella | NA | Moraxella osloensis | 99% |
| | Escherichia coli | 2% | osloensis | | | |
| ICU11 | Serrita rubidaea | 91% | Serrita rubidaea | Enterobacterales | Serrita rubidaea | 97% |
| ICU12 | Klebsiella variicola | 85% | Klebsiella | Enterobacterales | Klebsiella variicola | 53% |
| | Klebsiella pneumoniae | 2% | variicola | Klebsiella | Klebsiella | 41% |
| | | | | pneumoniae | pneumoniae | |
| ICU13 | Acinetobacter | 80% | Acinetobacter | NA | Acinetobacter | 84% |
| | guillouiae | | guillouiae | | guillouiae | |
| | | | | | Serrita rubidaea | 9% |
| ICU31- | Klebsiella aerogenes | 79% | Klebsiella | Klebsiella | Klebsiella aerogen | 90% |
| 2 | | | aerogens | aerogens | | |
| | Citrobacter cronae | 4.80% | Citrobacter cronae | Escherichia coli | Citrobacter cronae | 9% |
| ICU38 | Citrobacter freundii | 97% | Citrobacter freundii | Enterobacterales | Citrobacter murliniae | 36% |
| | | | | | Citrobacter gillenii | 22% |
| | | | | | Citrobacter freundii | 17% |
| | | | | | Citrobacter braakii | 13% |

NA: No amplicon detected

The performance of the method of the present disclosure in the identification of the phenotypic resistance and resistance genes in specimens was compared with FilmArray BCID2. As shown in Table 9, the method of the present disclosure could identify nearly all the resistance genes that could correspond to the phenotypic resistance detected by clinical blood culture and antimicrobial susceptibility testing (AST). In comparison, the FilmArray BCID2 only detected a limited number of the resistance genes.

TABLE 9
Comparison of phenotypic resistance and resistance genes identified by the method
of the present disclosure (i.e., TCDC protocol) and the FilmArray BCID2

| | Pathogen | Resistance | | |
|---|---|---|---|---|
| Sample | identification | identified | FilmArray | |
| No. | by blood culture | by AST | BCID2 | TCDC Protocol |
| 1 | Klebsiella | AM, SAM, TZP, CZ, | CTX-M | aadA16, aph (3′)-Ia, aph (6)-Id, aph |
| | pneumoniae | CTX, FEP, CIP, | OXA-48-like | (3″)-Ib, blaOXA-48, blaSHV-1, |
| | | LVX, SXT, MEM, | | blaCTX-M-15, blaTEM-1C, fosA, |
| | | ETP, IPM | | aac (6′)-Ib-cr, qnrB6, ARR-3, tet (A), |
| | | | | tet (D), OqxA, OqxB, qacE, dfrA7, |
| | | | | dfrA27, sul1, sul2 |
| 2-1 | Citrobacter freundii | AM, SAM, CZ, CMZ | ND | blaCMY-124, qnrB13 |
| 2-2 | Serratia marcescens | AM, SAM, CZ, CMZ | ND | aac (6′)-Ic, blaSRT-2, tet (41) |
| 3 | Enterobacter cloacae | AM, SAM, CZ, | ND | blaMIR-2, fosA |
| | | CMZ, IPM | | |
| 5 | Escherichia coli | AM, CZ, CTX, FEP | ND | tet (B), mdf (A), blaCTX-M-3 |
| 6 | Escherichia coli | GM, AM, SAM, | KPC | aph (6)-Id, aph (3″)-Ib, ant (6)-Ia, aac |
| | | TZP, CZ, CMZ, | VanA/B | (3)-IId, aadA1, aadA2, aph (3′)-III, |
| | | CTX, FEP, SXT, | | aac (6′)-aph (2″), aac (6′)-Il, floR, |
| | | ETP, IPM | | cmlA1, blaTEM-1B, blaSHV-11, |
| | Klebsiella | GM, AM, SAM, | | blaKPC-2, blaCMY-2, sul2, sul3, |
| | pneumoniae | TZP, CZ, CMZ, | | dfrA12, fosA, VanHAX, VanC1XY, |
| | | CTX, FEP, CIP, | | mdf (A), erm (42), msr (C), tet (A), |
| | | LVX, ETP, IPM | | tet (L), tet (M), tet (S) |
| | Enterococcus | VA, TEC | | |
| | gallinarum | | | |
| 7 | Staphylococcus | P, OX, E, CIP | mecA/C | aac (6′)-aph (2″), aadD, aph (3′)-III, |
| | aureus | | and MREJ | ant (6)-Ia, blaZ, mecA, lnu (A), mph |
| | | | (MRSA) | (C), msr (A), qacA, tet (K) |
| 8 | Pseudomonas | GM, CIP, LVX, IPM, | ND | aadA3, aac (6′)-Ib3, aph (3′)-IIb, |
| | aeruginosa | TZP, SXT | | blaCARB-2, blaPAO, blaOXA-494, |
| | | | | fosA, sul1, qacE, crpP, catB7 |
| 9 | Escherichia coli | AM, CIP, LVX, SXT | ND | tet (A), aph (6)-Id, aph (3″)-Ib, |
| | | | | blaTEM-1B, aadA5, sul1, sul2, mph |
| | | | | (A), qacE, dfrA17, mdf (A) |
| 10 | Klebsiella | AM | ND | blaOKP-B-2, blaACT-6, OqxA, |
| | pneumoniae | | | OqxB, fosA |
| | Enterobacter | AM, SAM, CZ, CMZ | | |
| | cloacae | | | |
| 12 | Klebsiella | AM | ND | blaLEN22, fosA, OqxA, OqxB |
| | variicola | | | |
| 13 | Acinetobacter | SAM, GM, CIP, | ND | aph (3′)-VI, aph (3′)-VIb, aph (3′)-Ia, |
| | guillouiae | LVX, CAZ, FEP, | | aac (3)-IId, aph (6)-Id, blaNDM-1, |
| | | IPM, MEM, TZP, | | blaOXA-274, blaOXA-58, tet (39), |
| | | SXT | | sul2 |
| 14 | Serratia marcescens | AM, SAM, CZ, | VIM | aac (6′)-Ic, ant (2″)-Ia, aac (6′)-Ib3, |
| | | CMZ, GM, TZP, | | aph (3′)-Ia, blaSRT-2, blaVIM-1, |
| | | CTX, FEP, CIP, | | blaOXA-10, qnrS1, tet (41), sul1, |
| | | LVX, ETP, IPM | | qacE, catB3 |
| 15 | Klebsiella | AM, SAM, TZP, CZ, | CTX-M | aph (6)-Id, aph (3″)-Ib, blaTEM-67, |
| | pneumoniae | CMZ, CTX, FEP, | KPC | blaCTX-M-14, blaCTX-M-65, |
| | | CIP, LVX, SXT, | | blaKPC-2, blaSHV-11, tet (A), sul2, |
| | | MEM, ETP, IPM | | fosA |
| 16 | Escherichia coli | AM, CZ, CTX, FEP, | CTX-M | aph (6)-Id, aph (3″)-Ib, blaCTX-M- |
| | | CIP, LVX | | 27, mph (A), sul1, sul2, tet (A), |
| | | | | qacE, mdf (A) |
| 17 | Klebsiella | AM | ND | blaSHV-1, OqxA, OqxB, fosA |
| | pneumoniae | | | |
| 18 | Pseudomonas | SXT | ND | aph (3′)-IIb, blaPAO, blaOXA-488, |
| | aeruginosa | | | catB7, fosA |
| 19 | Serratia marcescens | AM, AN, SAM, CZ | ND | aac (6′)-Ic, blaSRT-2, tet (41) |
| 20 | Escherichia coli | AM, CZ, CTX, FEP | CTX-M | blaTEM-1B, blaCTX-M-27, mdf (A) |
| 21 | Group B | CC | ND | aph (3′)-lll, ant (6)-Ia, erm (B), mre |
| | Streptococcus | | | (A), tet (M) |
| 22-1 | Escherichia coli | AM, CIP, LVX | ND | blaTEM-1B, mdf (A) |
| 22-2 | Pseudomonas | IPM, MEM, SXT | VanA/B | aph (3′)-llb, blaIPO, blaOXA-50, |
| | aeruginosa | | | catB7, crpP, fosA |
| 23 | Escherichia coli | AM, SXT | ND | aph (3″)-lb, aph (6)-ld, blaTEM-1B, |
| | | | | dfrA14, mdf (A), sul2 |
| 24 | Escherichia coli | AM | ND | blaTEM-1B, mdf (A), tet (B) |
| 25 | Escherichia coli | GM, AM | ND | blaTEM-1B, aac (3)-lld, mdf (A) |
| 26 | Escherichia coli | No resistance | ND | mdf (A) |
| 27 | Escherichia coli | AM, SAM, CZ, | ND | aph (6)-ld, aph (3″)-lb, blaCMY-2, |
| | | CMZ, CTX | | mdf (A), tet (A), sul2, floR |
| 28 | Klebsiella | AM | ND | blaSHV-11, fosA, OqxA, OqxB |
| | pneumoniae | | | |
| 29 | Klebsiella | GM, AN, CMZ, AM, | CTX-M | aac (3)-lld, aph (3″)-lb, aph (6)-ld, |
| | pneumoniae | SAM, TZP, CZ, | KPC | aadA1, rmtB, aac (6′)-lb-cr, catB3, |
| | | CTX, FEP, CIP, | NDM | blaTEM-67, blaCTX-M-14, blaSHV- |
| | | LVX, SXT, MEM, | | 11, blaTEM-1B, blaOXA-1, |
| | | ETP, IPM | | blaNDM-1, blaKPC-2, dfrA14, sul1, |
| | | | | sul2, fosA, qacE, qnrB1, tet (A) |
| 30 | Enterococcus | P, VA, TEC | VanA/B | aac (6′)-aph (2″), aac (6″)-li, aph (3′)- |
| | faecium | | | lll, ant (6)-la, dfrG, VanHAX, msr |
| | Candida albicans | NA | | (C), tet (M), tet (L) |
| 31-1 | Enterococcus | P, GMS, VA, TEC | VanA/B | VanHAX, aac (6′)-Ii, msr (C), dfrG, |
| | faecium | | | erm (B), ant (6)-Ia, aac (6′)-aph (2″), |
| | | | | cat (pC194), aph (3′)-III, ant (6)-Ia |
| 31-2 | Klebsiella | AM, SAM, CZ | NA | FosA |
| | aerogenes | | | |
| 32 | Proteus mirabilis | SXT | mecA/C | aph (6)-Id, aph (3″)-Ib, aadA2, aac |
| | Klebsiella | GM, AM, SAM, CZ, | | (3)-IId, aph (3′)-Ia, aac (6′)-aph (2″), |
| | pneumoniae | CTX, CMZ | | aadD, aph (3′)-IIIa, ant (6)-Ia, cat, |
| | Staphylococcus | NA | | floR, cat (pC221), OqxA, OqxB, |
| | epidermidis | | | blaDHA-1, blaTEM-1B, blaSHV-11, |
| | | | | blaZ, sul1, sul2, dfrA1, fosA, vga |
| | | | | (A)LC, mph (A), erm (C), qacA, |
| | | | | qnrB4, tet (A) |
| 33 | Escherichia coli | AM, SAM, CZ, CIP, | CTX-M | blaCTX-M-55, mdf (A) |
| | | LVX, CTX, FEP | | |
| 34 | Klebsiella | CMZ, AM, SAM, | ND | aph (3′)-Ia, aadA2, aac (3)-IId, aph |
| | pneumoniae | TZP, CZ, CTX, SXT | | (6)-Id, floR, blaSHV-65, blaTEM- |
| | | | | 1B, blaDHA-1, sul1, sul2, dfrA12, |
| | | | | fosA, mph (A), qacE, qnrB4, OqxA, |
| | | | | OqxB |
| 35 | Klebsiella | GM, CMZ, AM, | CTX-M | aph (6)-Id, aph (3″)-Ib, aac (3)-IId, |
| | pneumoniae | SAM, TZP, CZ, | KPC | blaKPC-2, blaSHV-11, blaTEM-1B, |
| | | CTX, MEM, ETP, | | blaCTX-M-14, sul2, fosA |
| | | IPM, FEP, CIP, LVX | | |
| 36 | Acinetobacter | SAM, AN, LVX, | ND | armA, aadA1, aac (6′)-Ib3, aph (3′)- |
| | baumannii | FEP, GM, CIP, CAZ, | | Ia, aph (6)-Id, aph (3″)-Ib, aadA24, |
| | | IPM, MEM, TZP, | | aac (3)-Ia, catB8, blaADC-25, |
| | | SXT | | blaOXA-23, blaTEM-1D, blaOXA-66, |
| | | | | sul1, mph (E), msr (E), qacE, tet (B) |
| 37 | Citrobacter | AM | ND | blaCKO-1 |
| | cronae | | | |
| 38 | Acinetobacter | SAM, AN, LVX, | ND | aph (3′)-Ia, aadA1, aac (3)-Ia, armA, |
| | baumannii | FEP, GM, CIP, CAZ, | | aac (6′)-Ib3, aph (6)-Id, aph (3″)-Ib, |
| | | IPM, MEM, TZP, | | catB8, blaOXA-23, blaOXA-66, |
| | | SXT | | blaTEM-1D, blaADC-25, sul1, mph |
| | | | | (E), msr (E), qacE, tet (B) |
| 40 | Stenotrophomonas | CAZ | ND | aph (3″)-IlC, aac (6′)-lz |
| | maltophilia | | | |
| 41 | Enterococcus | P, VA, TEC | VanA/B | aph (3′)-lll, ant (6)-Ia, aac (6′)-li, Inu |
| | faecium | | | (B), Isa (E), dfrG, VanHAX, msr (C), |
| | | | | tet (M), tet (L) |

AM: Ampicillin;
AN: Amikacin;
CAZ: Ceftazidime;
CC: clindamycin;
CIP: Ciprofloxacin;
CMZ: Cefmetazole;
CTX: Cefotaxime;
CZ: Cefazolin;
DAP: Daptomycin;
E: Erythromycin;
ETP: Ertapenem;
FEP: Cefepime;
GM: Gentamicin;
GMS: Gentamicin-Syn;
IPM: Imipenem;
LZD: Linezolid;
LVX: Levofloxacin;
MEM: Meropenem;
OX: oxacillin;
P: Penicillin;
SAM: Ampicillin-sulbactam;
SXT: Trimethoprim/Sulfamethoxazole;
TEC: Teicoplanin;
TGC: Tigecycline;
TZP: Piperacillin/Tazobactam;
VA: Vancomycin
NA: Not applicable
ND: Not detected

From the above, these data reveal that the method of the present disclosure can be used for the rapid identification of bacterial species and can reach 20× coverage depths of sequence within 2 to 4 hours of the sequencing time, thereby arriving at genome assembly, resistance genes detection, and antimicrobial susceptibility prediction. By employing immobilized adsorption, the system and method of the present disclosure can be used to obtain high-quality bacterial DNA by removal of non-target nucleic acid from humans or other sources in blood culture specimens. The extracted high-quality bacterial DNA may be subjected to rapid sequencing using the Nanopore sequencing platform to generate long sequence reads, which may be further analyzed using the bioinformatics pipelines to identify the species of bacteria and resistance genes.

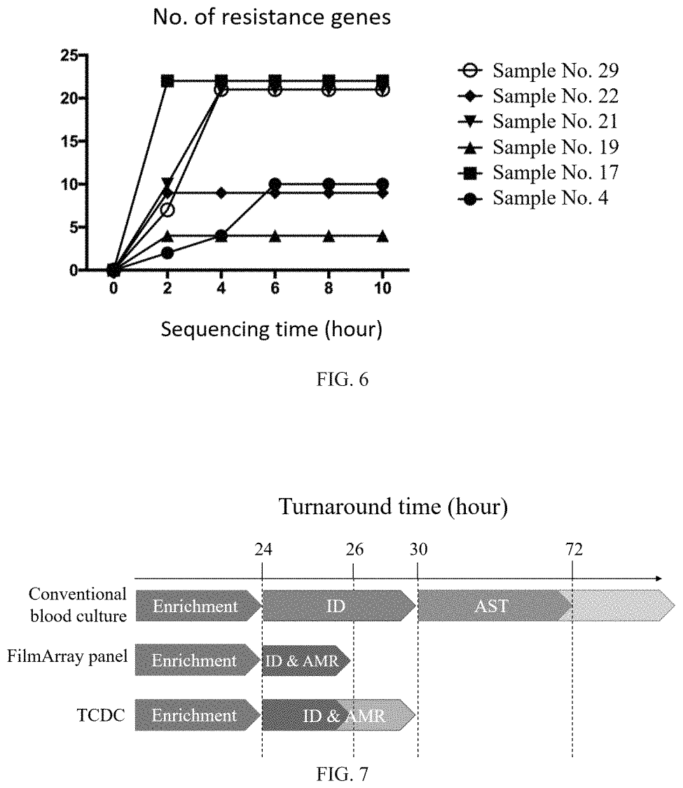

In comparison with conventional microbial culture followed by antimicrobial susceptibility testing, which requires a turnaround time of more than 3 days (FIG. 7), the blood culture specimens pretreated with the immobilized adsorption of the present disclosure for 2 hours can be subjected to Nanopore sequencing, and the pathogen and the resistance genes therein can be identified within 2 to 6 hours. In other words, by the system and method of the present disclosure, the information necessary to select a suitable antibiotic can be obtained only within 4 to 10 hours. Also, in comparison with the commercially available system for rapid detection, such as GeneXpert and FilmArray, the system and method of the present disclosure can be used to identify relatively various bacterial species and resistance genes, indicating the increased applicability for identification.

Hence, the present disclosure provides relevant information to timely select effective antimicrobials, thereby assisting in improving the cure rate of the diseases and curbing the emergence and spread of bacterial strains with resistance resulting from empirical use of non-effective antimicrobials.

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined to form a further embodiment. A method disclosed herein may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

What is claimed is:

1. A diagnostic system for identifying a target microorganism and/or a resistance gene in a sample, comprising:
a cell lysis unit configured to lyse a non-target cell in the sample, wherein the target microorganism and the non-target cell originate from different species;
a target nucleic acid enrichment unit equipped with an immobilized adsorption device, connected to the cell lysis unit, and configured to deplete a nucleic acid of the lysed non-target cell, thereby enriching a nucleic acid of the target microorganism in the sample;
a sequencing unit connected to the target nucleic acid enrichment unit and configured to sequence the enriched nucleic acid of the target microorganism; and
a sequence analysis unit connected to the sequencing unit and configured to receive sequencing data generated by the sequencing unit and to compare the sequencing data with a microbial genome database and/or a resistance gene database, thereby producing an identification result of the target microorganism and/or the resistance gene carried by the target microorganism.

2. The diagnostic system according to claim 1, wherein the cell lysis unit comprises a non-ionic surfactant selected from the group consisting of saponin, Tween, Triton, polyoxyethylene (10) oleyl ether, polyol, a polyoxyethylene-polyoxypropylene copolymer, polyoxyethylene ether, alkyl ethanolamide, glucoside, fatty alcohol, and any combination thereof.

3. The diagnostic system according to claim 1, wherein the immobilized adsorption device comprises a solid phase adsorbent selected from the group consisting of a silica magnetic bead, a silica bead, a column extraction membrane, an alkyl-bonded silica gel, a biochar, a cellulose, an anion exchange resin, and any combination thereof.

4. A method for enriching a target nucleic acid in a sample, comprising:
providing the sample including a target microorganism and a non-target cell, wherein the target microorganism and the non-target cell originate from different species;
lysing the non-target cell by a cell lysis unit of a diagnostic system to release a non-target nucleic acid from the non-target cell; and
depleting the non-target nucleic acid by a target nucleic acid enrichment unit of the diagnostic system, thereby enriching the target nucleic acid of the target microorganism in the sample.

5. The method according to claim 4, wherein the cell lysis unit comprises a non-ionic surfactant, and the lysis of the non-target cell comprises adding the non-ionic surfactant to the sample.

6. The method according to claim 4, wherein the target nucleic acid enrichment unit comprises an immobilized adsorption device containing a solid phase adsorbent, and the depletion of the non-target nucleic acid comprises:
contacting the sample with the solid phase adsorbent to bind the non-target nucleic acid; and
removing the solid phase adsorbent, thereby enriching the target nucleic acid in the sample.

7. The method according to claim 4, wherein the enriched nucleic acid has at least 2,000 nucleotides in length.

8. The method according to claim 4, which results in at least a 10-fold enrichment of the target nucleic acid originally comprised within the sample.

9. The method according to claim 4, wherein the target nucleic acid is selected from the group consisting of a pathogenic nucleic acid, a microbial nucleic acid, a bacterial nucleic acid, a viral nucleic acid, a fungal nucleic acid, an algae nucleic acid, a protozoan nucleic acid, a parasitic nucleic acid, and any combination thereof.

10. The method according to claim 4, wherein the target nucleic acid is a bacterial nucleic acid.

11. The method according to claim 4, wherein the non-target cell originates from a eukaryotic host.

12. The method according to claim 11, wherein the eukaryotic host is a mammalian host.

13. The method according to claim 4, wherein the sample is an environmental sample or a biological sample obtained from a host suffering or suspected of suffering from an infectious disease.

14. The method according to claim 13, wherein the infectious disease is bacteremia, sepsis, or pneumonia.

15. The method according to claim 13, wherein the biological sample is selected from the group consisting of blood, serum, plasma, urine, sputum, saliva, cerebrospinal fluid, interstitial fluid, mucous, sweat, stool extract, fecal matter, synovial fluid, tears, semen, peritoneal fluid, nipple aspirates, milk, vaginal fluid, and any combination thereof, and the environmental sample is selected from the group consisting of dust, soil, water, air, an artificial water system, food, and any combination thereof.

16. The method according to claim 4, further comprising:
sequencing the enriched nucleic acid by a sequencing assay to generate sequencing data; and
comparing the sequencing data with a microbial genome database and/or a resistance gene database, thereby producing an identification result of the target microorganism and/or the resistance gene carried by the target microorganism.

17. The method according to claim 16, wherein the sequencing assay is selected from the group consisting of a next-generation sequencing assay, a high-throughput sequencing assay, an Illumina sequencing assay, a Nanopore sequencing assay, a PacBio sequencing assay, a Sanger sequencing assay, and any combination thereof.

18. The method according to claim 16, further comprising extracting the enriched nucleic acid from the sample prior to the sequencing.

19. The method according to claim 16, wherein the sequencing of the enriched nucleic acid comprises generating the sequencing data with at least 20 times the genome size of the target microorganism.

20. A method for enriching a target nucleic acid in a sample, comprising:
providing the sample including a target microorganism and a non-target cell, wherein the target microorganism and the non-target cell originate from different species;
adding a non-ionic surfactant to the sample, wherein the non-ionic surfactant is selected from the group consisting of saponin, Tween, Triton, polyoxyethylene (10) oleyl ether, polyol, a polyoxyethylene-polyoxypropylene copolymer, polyoxyethylene ether, alkyl ethanolamide, glucoside, fatty alcohol, and any combination thereof;
contacting the sample with a solid phase adsorbent to bind free nucleic acids in the sample, wherein the solid phase adsorbent is selected from the group consisting of a silica magnetic bead, a silica bead, a column extraction membrane, an alkyl-bonded silica gel, a biochar, a cellulose, an anion exchange resin, and any combination thereof; and
removing the solid phase adsorbent, thereby enriching the target nucleic acid in the sample.
